(19) United States
(12) Patent Application Publication
(10) Pub. No.: US 2023/0139632 A1
(43) Pub. Date: May 4, 2023

(54) METHOD AND DEVICE FOR PURIFYING VISCOUS AND/OR SOLID ISOCYANATES FROM A PRODUCT FLOW FROM ISOCYANATE SYNTHESIS

(71) Applicant: Covestro Deutschland AG, Leverkusen (DE)

(72) Inventors: Joerg Morawski, Leverkusen (DE); Rainer Bellinghausen, Odenthal (DE)

(21) Appl. No.: 17/909,448

(22) PCT Filed: Mar. 15, 2021
(86) PCT No.: PCT/EP2021/056479

§ 371 Date: Sep. 6, 2022

(30) Foreign Application Priority Data
Mar. 18, 2020 (EP) 20164062.0

(51) Int. Cl. C07C 263/20

(52) U.S. Cl. CPC C07C 263/20

(57) ABSTRACT

The present invention relates to a process for the purification of a product stream from isocyanate synthesis, wherein the isocyanate converted to the gas phase from the product stream is continuously deposited on a rotating cooled roller and removed from the roller. The present invention further relates to an apparatus for the purification of a product stream from isocyanate synthesis, wherein the apparatus has both a heated roller and a condensation roller.

The present invention relates to a process for the purification of a product stream from isocyanate synthesis, wherein the isocyanate converted to the gas phase from the product stream is continuously deposited on a rotating cooled roller and removed from the roller. The present invention further relates to an apparatus for the purification of a product stream from isocyanate synthesis, wherein the apparatus has both a heated roller and a condensation roller.

### PRIOR ART

The distillation of viscous or solid isocyanates from product streams from isocyanate synthesis constitutes a particular challenge for apparatus technology since conventional thin-film evaporators or short-path evaporators require a certain flowability of the residue in order to ensure the discharge thereof. At the same time, the condensed isocyanate distillate must also have a sufficient flowability since otherwise discharge can only be guaranteed by regular melting off or by high condenser temperatures.

Various processes and apparatuses for preparing pure isocyanate from product streams of isocyanate synthesis have already been described in the literature, with high condenser temperatures being used in order to keep the solid isocyanate liquid in the condenser.

For instance, DE 2035731 describes a process for the distillation of isocyanate residues, in which an evaporation condenser operated with chlorobenzene is used in order to avoid problems associated with deposits caused by the solid isocyanate in the condenser. The boiling point of chlorobenzene ensures that the solid isocyanate is obtained in liquid form. This liquid isocyanate is then crystallized and flaked on a flaking roller. A disadvantage of this process is the high temperature required for condensation, which limits the vacuum. Due to the limited vacuum, a higher evaporator temperature has to be used, which can lead to undesired side reactions of the isocyanate and in this way lowers the yield. Moreover, at these high temperatures the isocyanate present in the gas phase does not fully condense, meaning that a portion of the isocyanate is located in the offgas and must be removed by a scrubber. This further reduces the yield. In addition, the scrubber generates an additional pressure drop, which further limits the achievable vacuum. Furthermore, the process disclosed in this document has high apparatus complexity, since an evaporator condenser, a condenser for evaporated chlorobenzene, a scrubber and a flaking roller are needed in order to isolate the purified solid isocyanate from the product stream by distillation.

FR 987091 describes the use of a rotating cooled roller for the thickening of solutions, pastes or solids, these first being heated and then sprayed onto the roller while being expanded. As a result, volatile fractions, for example solvents and water, evaporate and the nonvolatile fractions are condensed and concentrated on the roller. Condensation of the volatile fractions, i.e. the distillate, on the cooled roller is undesirable in this process since this hinders the thickening.

WO 2012/011805 and US 2003/0062431 describe drum dryers in which the vapors arising during the drying are collected and fed into a conventional condenser. In the case of viscous or solid isocyanates, the condenser has to be operated with a sufficiently high temperature for the condensate to remain liquid and to avoid clogging. However, these high temperatures entail losses in yield and a complex offgas purification, since the gaseous isocyanate is no longer fully condensed at such high temperatures.

WO 2019/193073 describes the evaporation of a room temperature-solid isocyanate, in which, inter alia, a drum dryer may also be used. This document does not contain any references to suitable condensers.

There is therefore a need for apparatuses and processes for separating solid or viscous isocyanate from product streams of isocyanate synthesis, in which the solid or viscous isocyanate can be obtained with maximum purity and yield and at the same time apparatus complexity and energy expenditure are minimized.

### Problem Addressed by the Invention

An object of the present invention was accordingly that of providing a process and an apparatus for purifying a product stream from isocyanate synthesis, in which the apparatus complexity is to be minimized and purification is possible at low pressures and low temperatures. Furthermore, it should be possible in particular with the apparatus and the process to isolate solid or viscous isocyanates from the product stream in high purities and yields.

### Achievement of the Object

The object is achieved by a process for the purification of a product stream from isocyanate synthesis, comprising the following process steps:
a) applying a negative pressure to a working space by means of a negative pressure conduit connected to the working space,
b) supplying the product stream to at least one heated roller arranged in the working space via a product feed conduit which is heatable and connected to the working space,
c) converting the isocyanate present in the product stream to the gas phase on the at least one heated roller and discharging the residue collected on the at least one heated roller via at least one residue discharge conduit connected to the working space and
d) condensing the isocyanate converted to the gas phase at a condenser arranged in the working space and continuously discharging the isocyanate deposited on the condenser via at least one isocyanate discharge conduit connected to the working space and optionally to the condenser,
   characterized in that
   the condenser is a rotating cooled roller at which at least one isocyanate removing device is arranged for the continuous removal of the isocyanate deposited from the gas phase on the condenser.

Furthermore, the object is achieved by an apparatus 1 for the purification of a product stream from isocyanate synthesis, comprising
at least one working space 2,
at least one product feed conduit 3 which is heatable and connected to the working space 2 for supplying the product stream into the working space,
at least one heated roller 4 arranged in the working space,
a condenser 5 arranged in the working space,
at least one isocyanate discharge conduit 6 connected to the working space 2 and optionally to the condenser 5 for discharging the isocyanate deposited on the condenser,
at least one residue discharge conduit 7 connected to the working space 2 for discharging the residue collected on the at least one heated roller and
a negative pressure conduit 8 preferably connected to the working space 2 at the level of the condenser 5 for applying a negative pressure to the working space,
   characterized in that
   the condenser is a rotating cooled roller at which at least one isocyanate removing device 9 is arranged for the continuous removal of the isocyanate deposited on the condenser 5.

The invention is based on the finding that the evaporation of the isocyanate from the product stream and the condensation thereof in just a single apparatus makes it possible to carry out the process at lower pressures since undesirable pressure drops via pipelines connecting the evaporation unit with the condenser do not arise. As a result, the evaporation can be conducted at lower temperatures, which reduces undesirable side reactions, increases the yield of isocyanate and achieves an improved energy balance. Since the isocyanate does not have to be obtained in liquid form at the condenser in order to guarantee discharge, this condenser can be operated at lower temperatures, as a result of which the yield is increased and complex offgas purification operations for removing isocyanate residues are avoided. The use of a scraper to remove the isocyanate deposited in solid form on the roller means that regular melting off of the condenser is unnecessary and hence a continuous process regime is also enabled.

The invention especially relates to the following embodiments:

According to a first embodiment, the present invention relates to a process for the purification of a product stream from isocyanate synthesis, comprising the following process steps:
a) applying a negative pressure to a working space 2 by means of a negative pressure conduit 8 connected to the working space 2,
b) supplying the product stream to at least one heated roller 4 arranged in the working space 2 via a product feed conduit 3 which is heatable and connected to the working space 2,
c) converting the isocyanate present in the product stream to the gas phase on the at least one heated roller 4 and discharging the residue collected on the at least one heated roller 4 via at least one residue discharge conduit 7 connected to the working space 2 and
d) condensing the isocyanate converted to the gas phase at a condenser 5 arranged in the working space 2 and continuously discharging the isocyanate deposited on the condenser 5 via at least one isocyanate discharge conduit 6 connected to the working space 2 and optionally to the condenser 5,
   characterized in that
   the condenser 5 is a rotating cooled roller at which at least one isocyanate removing device 9 is arranged for the continuous removal of the isocyanate deposited from the gas phase on the condenser 5.

According to a second embodiment, the present invention relates to a process according to embodiment 1, wherein the product stream has been obtained by phosgenation of an amine, by phosgenation of an amine hydrochloride, by phosgenation of a carbamate salt, by the urea process or by reaction of an amine with dialkyl carbonates.

According to a third embodiment, the present invention relates to a process according to embodiment 1 or 2, wherein the at least one heated roller 4 rotates.

According to a fourth embodiment, the present invention relates to a process according to any of the preceding embodiments, wherein the product stream is supplied onto the at least one heated roller 4 from above.

According to a fifth embodiment, the present invention relates to a process according to any of the preceding embodiments, wherein the product stream has a temperature which is above the melting point of the isocyanate to be converted to the gas phase and not more than 10 kelvin above the evaporation temperature of this isocyanate.

According to a sixth embodiment, the present invention relates to a process according to any of the preceding embodiments, wherein the at least one heated roller 4 has a temperature which is 5 to 50 kelvin above the evaporation temperature of the isocyanate to be converted to the gas phase at the pressure prevailing in the working space 2.

According to a seventh embodiment, the present invention relates to a process according to any of the preceding embodiments, wherein the residue formed on the at least one heated roller 4 is removed with the aid of a residue removing device 10, in particular a scraper, or at least one residue side discharge 10.

According to an eighth embodiment, the present invention relates to a process according to any of the preceding embodiments, wherein the isocyanate deposited continuously from the gas phase is removed from the at least one rotating cooled roller 5 with the aid of a scraper 9.

According to a ninth embodiment, the present invention relates to a process according to any of the preceding embodiments, wherein the rotating cooled roller 5 has a temperature which is 5 to 30 kelvin below the melting point of the isocyanate converted to the gas phase at the pressure prevailing in the working space 2.

According to a tenth embodiment, the present invention relates to a process according to any of the preceding embodiments, wherein the working space 2 has a temperature which is 2 to 20 kelvin above the evaporation temperature of the isocyanate converted to the gas phase at the pressure prevailing in the working space 2.

According to an eleventh embodiment, the present invention relates to a process according to any of the preceding embodiments, wherein the isocyanate converted to the gas phase has a melting point of 120 to 135° C., determined in accordance with DIN 51556:1963-07.

According to a twelfth embodiment, the present invention relates to a process according to any of the preceding embodiments, wherein the isocyanate converted to the gas phase is selected from aliphatic and/or aromatic diisocyanates, preferably from aromatic diisocyanates, in particular from naphthylene 1,5-diisocyanate.

According to a thirteenth embodiment, the present invention relates to a process according to any of the preceding embodiments, wherein the process is conducted at a pressure of 0.1 to 5 mbar, preferably at 0.5 to 5 mbar, in particular at 1 to 5 mbar.

According to a fourteenth embodiment, the present invention relates to an apparatus 1 for the purification of a product stream from isocyanate synthesis, comprising
at least one working space 2,
at least one product feed conduit 3 which is heatable and connected to the working space 2 for supplying the product stream into the working space 2,
at least one heated roller 4 arranged in the working space 2,
a condenser 5 arranged in the working space 2,
at least one isocyanate discharge conduit 6 connected to the working space 2 and optionally to the condenser 5 for discharging the isocyanate deposited on the condenser 5,
at least one residue discharge conduit 7 connected to the working space 2 for discharging the residue collected on the at least one heated roller 4 and
a negative pressure conduit 8 preferably connected to the working space 2 at the level of the condenser 5 for applying a negative pressure to the working space 2,
   characterized in that
   the condenser 5 is a rotating cooled roller at which at least one isocyanate removing device 9 is arranged for the continuous removal of the isocyanate deposited on the condenser 5.

According to a fifteenth embodiment, the present invention relates to an apparatus according to embodiment 14, wherein the isocyanate product feed conduit 3 is located above the at least one heated roller 4.

According to a sixteenth embodiment, the present invention relates to an apparatus according to embodiment 14 or 15, wherein in the working space at least two, in particular exactly two, heated rollers 4 are arranged preferably substantially parallel next to one another.

According to a seventeenth embodiment, the present invention relates to an apparatus according to any of embodiments 14 to 16, wherein at the at least one heated roller 4 there is arranged at least one residue removing device 10, in particular a scraper, or at least one residue side discharge 10 for removing the residue collected on the at least one heated roller 4.

According to an eighteenth embodiment, the present invention relates to an apparatus according to any of embodiments 14 to 17, wherein the condenser 5 is arranged above the at least one heated roller 4.

According to a nineteenth embodiment, the present invention relates to an apparatus according to any of embodiments 14 to 17, wherein the condenser 5 is arranged in a condensation chamber connected to the working space 2, wherein the connection between the working space 2 and the condensation chamber is preferably positioned laterally above the at least one heated roller 4 and opposite the product feed conduit 3.

According to a twentieth embodiment, the present invention relates to an apparatus according to any of embodiments 14 to 19, wherein the working space 2 has an outer jacket 12 for heating the working space 2 by means of heat, in particular by means of steam and/or by means of oil.

### Detailed Description of the Invention

### DESCRIPTION OF THE FIGURES

The invention is elucidated in more detail with reference to FIGS. 1 to 4, in which various embodiments variants of the invention are illustrated:

FIG. 1 shows an apparatus 1 according to the invention, comprising a pear-shaped working space 2, an isocyanate product feed conduit 3, which leads into the working space 2 above the at least one heated roller 4 and ends immediately before the vertical axis of this clockwise rotating heated roller 4, and a condenser 5 arranged above the heated roller 4 and rotating clockwise. An isocyanate discharge conduit 6 is arranged directly at the condenser 5. At the end of this isocyanate discharge conduit there is a drop shaft 6′ through which the pure isocyanate is guided into a silo 15 and collected there. The negative pressure conduit 8 for generating a vacuum in the working space 2 is located at the level of the condenser 5. Two isocyanate removing devices 9, preferably in the form of scrapers, are further arranged at the condenser 5 in such a way that the pure isocyanate scraped off can be removed from the working space via the isocyanate discharge conduit 6. A residue removing device 10 in the form of a scraper is located at the heated roller 4 for removing the residue collected on the heated roller 4. The residue removed by means of the scraper 10 falls down into the working space, is guided into a silo 14 via a residue discharge conduit 7 in the form of a drop shaft and is collected in said silo. The scraper 10 is located at the heated roller 4 immediately before the end of the isocyanate product feed conduit 3. The isocyanate product feed conduit 3 has an outer jacket 13 which is connected to a heating medium feed conduit 13′ and a heating medium discharge conduit 13″, via which used heating medium can be discharged. The working space 2 also has an outer jacket 12 which is connected to a heating medium feed conduit 16 at the upper end of the working space and heating medium discharge conduit 17 at the lower end of the working space 2.

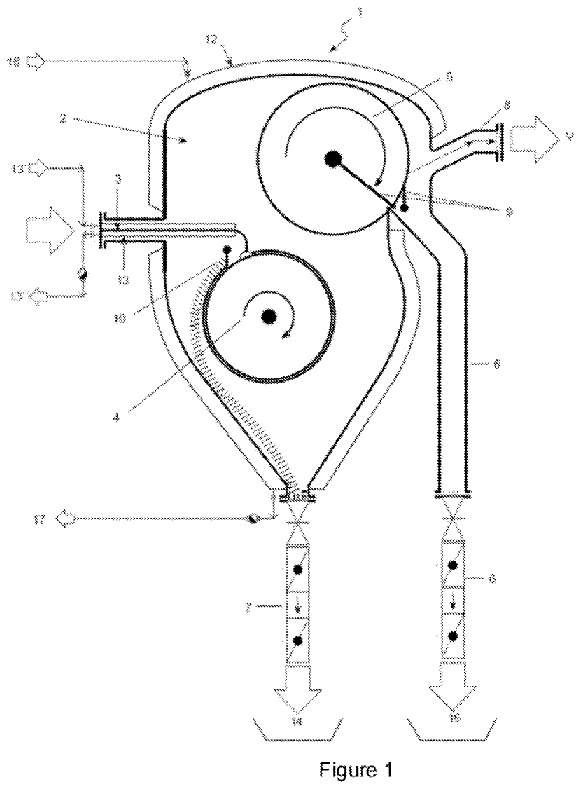

FIG. 2 illustrates an apparatus 1 according to the invention in analogous fashion to FIG. 1, wherein this apparatus has two heated rollers 4 and 4′ arranged in parallel in a plane in the working space 2. The first heated roller 4 rotates clockwise, the second heated roller 4′ rotates counterclockwise. The isocyanate product feed conduit leads into the working space 2 above the two heated rollers 4 and 4′ and ends above the gap between the two heated rollers. A residue removing device 10 in the form of a scraper is located at each of the two heated rollers 4 and 4′ for removing the residue collected on the heated rollers 4 and 4′. The scrapers 10 at the heated rollers 4 and 4′ are each located between the vertical and the horizontal axis of the respective heated roller before the end of the isocyanate product feed conduit 3.

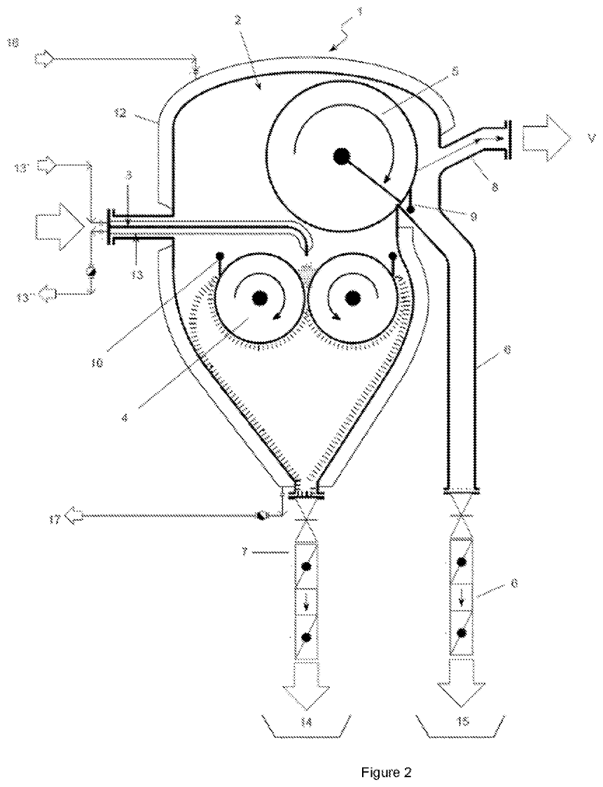

FIG. 3 shows an apparatus 1 according to the invention in which the condenser 5 is arranged in a condensation chamber connected to the working space 2, wherein the connection between the working space 2 and the condensation chamber is preferably positioned laterally above the at least one heated roller 4 and opposite the product feed conduit 3. The condensation chamber in this case is connected via a connection 18 to the rest of the working space, which is of pear-shaped form. The negative pressure conduit 8 is arranged at the end of the condensation chamber for generation of a vacuum in the working space 2. The condenser 5 rotates clockwise and on the vertical axis and just beyond the vertical axis has two isocyanate removing devices 9 in the form of scrapers. Directly beneath the isocyanate removing device 9 arranged on the vertical axis there is located the isocyanate discharge conduit 6, via which the pure isocyanate scraped off from the condenser 5 is guided into the drop shaft 6′ and is then collected in a silo 15. The pear-shaped part of the working space 2 is connected to an isocyanate product feed conduit 3 which leads into the working space 2 above the at least one heated roller 4 and ends immediately before the vertical axis of this heated roller 4. A residue removing device 10 in the form of a scraper is located at the heated roller 4 for removing the residue collected on the heated roller 4. The residue removed by means of the scraper 10 falls down into the working space, is guided into a silo 14 via a residue discharge conduit 7 in the form of a drop shaft and is collected in said silo. The scraper 10 is located at the heated roller 4 immediately before the end of the isocyanate product feed conduit 3. The isocyanate product feed conduit 3 has an outer jacket 13 which is connected to a heating medium feed conduit 13′ and a heating medium discharge conduit 13″, via which used heating medium can be discharged. The working space 2 also has an outer jacket 12 which is connected to a heating medium feed conduit 16 at the upper end of the working space and heating medium discharge conduit 17 at the lower end of the working space 2.

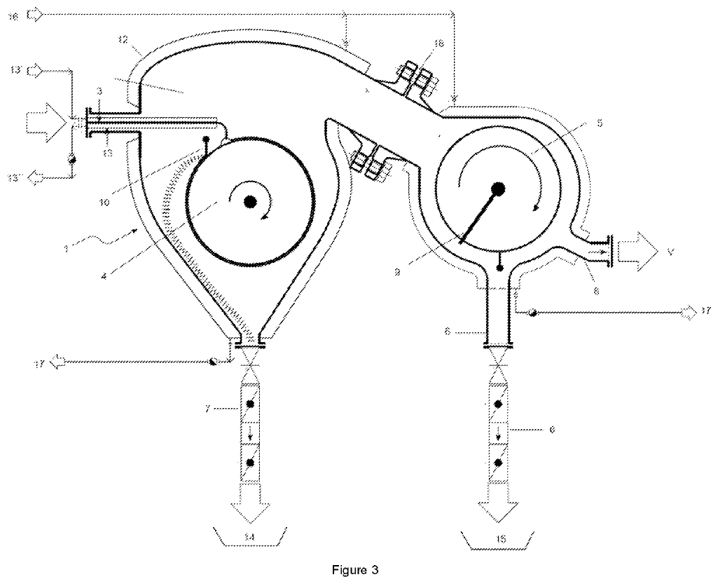

FIG. 4 illustrates an apparatus 1 according to the invention in analogous fashion to FIG. 3, wherein this apparatus has two heated rollers 4 and 4′ arranged in parallel in a plane in the pear-shaped part of the working space. The first heated roller 4 rotates clockwise, the second heated roller 4′ rotates counterclockwise. The isocyanate product feed conduit leads into the working space 2 above the two heated rollers 4 and 4′ and ends above the gap between the two heated rollers. A residue removing device 10 in the form of a scraper is located at each of the two heated rollers 4 and 4′ for removing the residue collected on the heated rollers 4 and 4′. The scrapers 10 at the heated rollers 4 and 4′ are each located between the vertical and the horizontal axis of the respective heated roller before the end of the isocyanate product feed conduit 3.

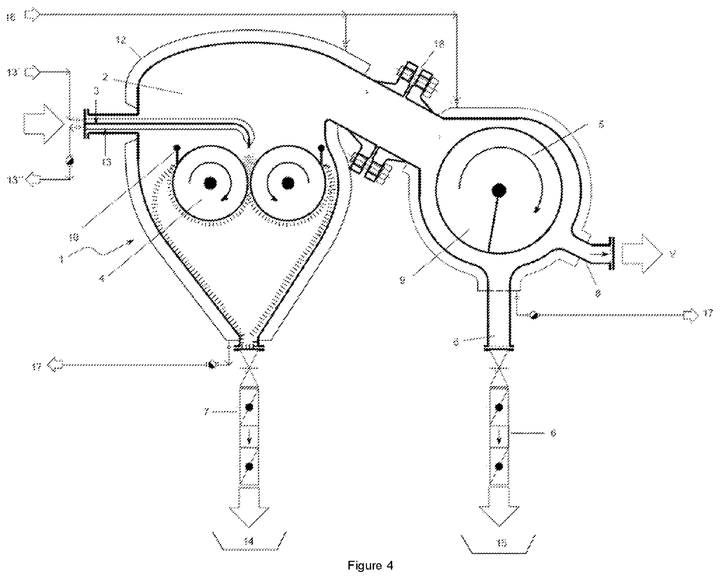

### PROCESS ACCORDING TO THE INVENTION

By means of the process according to the invention, product streams obtainable from isocyanate synthesis (hereinafter also referred to as crude isocyanate product stream) can be purified effectively and in a gentle manner. Since the pure isocyanate does not need to be obtained in liquid form, the process according to the invention is exceedingly suitable for the purification of isocyanates obtained in viscous or solid form at the condenser.

### Process Step a):

Economically relevant losses in yield typically arise during the preparation of the pure isocyanate from the crude isocyanate product stream as a result of the fact that the pure isocyanate to be isolated is subject to relatively long residence times in regions of relatively high temperature. The temperature stress leads to the formation of oligomers of the isocyanate (isocyanurate, carbodiimide, uretdione, etc.) which reduces the yield. The problem of losses in yield is typically solved by conducting the purifying distillation under high vacuum. Distillation under high vacuum leads to a lowering of the temperature level in the distillation apparatus, which reduces the tendency toward polymerization.

In step a) of the process according to the invention, therefore, a negative pressure is applied to a working space 2 by means of a negative pressure conduit 8 connected to the working space 2. The negative pressure applied in this process step preferably corresponds to the pressure at which the process according to the invention is conducted. The negative pressure can be generated using any apparatuses known to those skilled in the art, example using vacuum pumps or the like.

### Process Step b):

In process step b) of the process according to the invention, a product stream is supplied to at least one heated roller 4 arranged in the working space 2 via a product feed conduit 3 which is heatable and connected to the working space 2.

The crude isocyanate product stream to be purified by means of the process according to the invention may be obtained by various processes. Examples of suitable processes for preparing the crude isocyanate product stream include the phosgenation of an amine, the phosgenation of an amine hydrochloride or of a carbamate salt by what is known as the urea process or the reaction of the parent amine with dialkyl carbonates. The isocyanate is preferably prepared by phosgenation.

In the phosgenation, the corresponding primary amine or its hydrochloride is usually reacted with an excess of phosgene in a solvent or in the gas phase. The process is preferably conducted in a solvent.

The solvent used is typically a solvent which has a lower boiling point than the isocyanate to be purified, preferably chlorobenzene, o- or p-dichlorobenzene, trichlorobenzene, chlorotoluenes, chloroxylenes, chloroethylbenzene, chloronaphthalenes, chlorodiphenyls, methylene chloride, perchloroethylene, toluene, xylenes, hexane, decahydronaphthalene, diethyl isophthalate (DEIP) and other carboxylic esters, tetrahydrofuran (THF), dimethylformamide (DMF) and benzene, and mixtures thereof. Particular preference is given to using chlorobenzene and dichlorobenzene as solvents. The amine is preferably dissolved in the solvent prior to addition of the phosgene, with the amine preferably being used in a total amount of 1 to 50 percent by mass, especially 3 to 40 percent by mass, based in each case on the amount of amine and solvent.

The mixture obtained after the reaction (hereinafter referred to as reaction output) is usually in the form of a suspension. This suspension contains the isocyanate to be purified as a liquid, carbamyl chlorides which have not yet decomposed and possibly amine hydrochlorides and/or ureas as solids, residues of hydrogen chloride, excess phosgene, solvents and impurities and non-evaporable polymeric residue. These impurities and the polymeric residue are formed during the phosgenation by incomplete reaction or undesirable side reactions or parallel reactions.

First, the hydrogen chloride and excess phosgene are removed from the reaction output in one or more steps using processes known in the prior art. The solvent is then distilled off. The crude isocyanate product stream produced in this way is then purified by means of the process according to the invention in order to obtain the pure isocyanate.

If the phosgenation is conducted in the gas phase, the amine is preferably used in pure form. The reaction output containing the isocyanate is in this case quenched from the gas phase in an inert solvent. After removal of hydrogen chloride, excess phosgene and solvent, the crude isocyanate product stream obtained in this way is purified by means of the process according to the invention. The gas-phase phosgenation may possibly also be effected without the use of an additional solvent. In this case, the process according to the invention is performed on the crude isocyanate product stream after removal of hydrogen chloride and excess phosgene.

The hydrogen chloride, excess phosgene and solvent are therefore preferably substantially completely removed from the crude isocyanate product stream used in step b). The content of hydrogen chloride and phosgene is thus preferably less than 1000 ppm in each case. In addition, the solvent content is preferably below 1% by weight, preferably below 0.5% by weight and particularly preferably below 0.1% by weight, based on the total weight of the crude isocyanate product stream.

In step b), the product stream is preferably supplied onto a rotating heated roller 4. The heated roller may in this case rotate clockwise or counterclockwise. The heated roller 4 preferably rotates clockwise. When using more than one heated roller 4, the heated rollers preferably rotate in opposite directions, i.e., when using two heated rollers 4 and 4′, the first heated roller 4 rotates clockwise and the second heated roller 4′ rotates counterclockwise or vice versa.

The product stream introduced in process step b) is preferably supplied onto the at least one heated roller 4 from above. This ensures that the product stream comes into contact with the heated roller for a sufficiently long period of time in order to substantially fully evaporate the pure isocyanate and to condense the undesirable residues on the heated roller before these drop down and clog the residue discharge conduit 7. When only one heated roller 4 is used, therefore, the product stream is preferably supplied from above between the horizontal and the vertical axis of this heated roller, with preference being given to a supply—as viewed in the direction of rotation—immediately before the vertical axis of the heated roller 4. If more than one heated roller is used, for example two heated rollers 4 and 4′, the product stream is preferably supplied from above in such a manner that both heated rollers come into contact with the product stream equally. It is therefore advantageous, in the case of heated rollers 4 and 4′ arranged parallel next to one another at the same height, to supply the product stream from above into the gap formed between the heated rollers 4 and 4′ arranged in parallel.

In order to ensure supply of the crude isocyanate product stream in liquid form, the isocyanate product feed conduit 3 is heated. For this purpose, heating media, such as steam, oil, electric heating, heating tapes or the like are preferably used, these being continuously supplied and removed in order to guarantee a consistent temperature within the isocyanate product feed conduit 3. In this way, solidification of the crude isocyanate product stream and clogging of the product feed conduit are avoided. The isocyanate product feed conduit is preferably heated in this case such that the product stream has a temperature which is above the melting point of the pure isocyanate to be converted to the gas phase and not more than 10 kelvin above the evaporation temperature of this isocyanate. This ensures that the product stream on the one hand remains liquid in order to avoid clogging of the isocyanate product conduit, and yet on the other hand that a reduction in the yield as a result of undesired oligomerization of the pure isocyanate at excessively high temperatures is avoided.

### Process Step c):

In process step c) of the process according to the invention, the isocyanate/pure isocyanate present in the product stream is converted to the gas phase on the at least one heated roller 4. In addition, the residue collected on the at least one heated roller 4 is discharged via at least one residue discharge conduit 7 connected to the working space 2.

The at least one heated roller 4 preferably has a temperature which is 5 to 50 kelvin above the evaporation temperature of the isocyanate to be converted to the gas phase at the pressure prevailing in the working space 2. On the one hand, these temperatures provide substantially complete evaporation of the pure isocyanate to be converted to the gas phase. On the other hand, however, a reduction in the yield as a result of undesired oligomerization reactions arising at excessively high temperatures is avoided. When using more than one heated roller 4, for example two heated rollers 4 and 4′, it is advantageous according to the invention when both heated rollers have the temperature specified above.

A “substantially complete evaporation” of the isocyanate to be converted to the gas phase is preferably present when the distillation residue, based on its total weight including the pure isocyanate, consists of the pure isocyanate to an extent of not more than 20% by weight, more preferably to an extent of not more than 10% by weight, and particularly preferably not more than 5% by weight. Since the distillation residue can be mechanically removed from the roller, no pure isocyanate need remain in the residue to ensure the flowability thereof.

It is further preferable for the evaporation in process step c) to be carried out such that the distillation residue is no longer flowable and particularly preferably is in solid form. It is thus particularly easy to remove it from the roller and discharge it from the system.

Since flowability of the distillation residue is not a requirement, the process according to the invention is preferably carried out such that the product stream is not mixed with more than 10% by weight of flow agent, based on the total mass of the product stream, either prior to the start of process step c) nor during the performance thereof. It is particularly preferable for no flow agent to be used at all.

In this application, “flow agent” is understood to mean any substance which is in liquid form under the conditions present at the heated roller without evaporating. A flow agent has the function of keeping parts of the distillation residue that are in solid form flowable by embedding or dissolution. This is necessary when using falling-tube or short-path evaporators because clogging of the device by solid residues should be avoided. According to the invention, the use of a heated roller especially enables simple removal of even solid distillation residues. In particular, a “flow agent” which according to the invention is to be excluded from use or subject to restricted use is understood to mean bitumen.

The reside formed on the at least one heated roller 4 is preferably removed with the aid of a residue removing device 10, in particular a scraper, or at least one residue side discharge 10. The residue formed is preferably removed continuously in order to ensure that the surface of the heated roller 4 with which the isocyanate product stream comes into contact has a consistent temperature. This achieves substantially complete conversion of the pure isocyanate from the product stream to the gas phase. The residue formed on the at least one heated roller 4 is therefore particularly preferably removed immediately before the point on the heated roller 4 at which the isocyanate product stream is supplied. When using more than one heated roller, the residue is preferably removed on each of the heated rollers in the manner described above.

### Process Step d):

In process step d) of the process according to the invention, the pure isocyanate converted to the gas phase is condensed at a condenser 5 arranged in the working space 2. The pure isocyanate deposited on this condenser 5 is discharged via an isocyanate discharge conduit 6 connected to the working space 2 and optionally to the condenser 5.

The condenser 5 is a rotating cooled roller at which at least one isocyanate removing device 9 is arranged for the continuous removal of the pure isocyanate deposited from the gas phase on the condenser 5. On the one hand, the continuous removal enables the surface of the condenser to have a consistent temperature so that it is ensured that the pure isocyanate is substantially completely deposited from the gas phase on this condenser. On the other hand, the continuous removal avoids the formation of thick pure isocyanate layers on the condenser which can be completely removed only with difficulty. Due to the continuous evaporation of the crude isocyanate and the continuous removal of the pure isocyanate, the process according to the invention can therefore also be operated continuously when, as pure isocyanate, a viscous or solid pure isocyanate is deposited on the condenser. The condenser can be cooled with any coolants known to those skilled in the art, especially by means of monochlorobenzene (MCB) or cooling water.

The isocyanate continuously deposited from the gas phase is preferably removed from the at least one rotating cooled roller with the aid of a scraper 9. The removal by means of the scraper is preferably effected in such a way that the pure isocyanate scraped off is immediately guided into the isocyanate discharge conduit 6 in order to ensure that the pure isocyanate is completely removed from the working space and is not exposed to high temperatures for longer than is necessary. This avoids losses in yield.

The rotating cooled roller preferably has a temperature which is 5 to 30 kelvin below the melting point of the isocyanate converted to the gas phase at the pressure prevailing in the working space 2. This ensures substantially complete condensation of the pure isocyanate from the gas phase on the rotating cooled roller.

In order to avoid the pure isocyanate from condensing on the walls of the working space, the working space 2 preferably has a temperature which is 2 to 20 kelvin above the evaporation temperature of the isocyanate converted to the gas phase at the pressure prevailing in the working space 2. As a result, losses in yield due to deposits of the pure isocyanate in the working space and also time- and cost-intensive measures for cleaning the working space 2 are avoided.

The process according to the invention is particularly suitable for the purification of solid isocyanates from the crude isocyanate product stream. The isocyanate converted to the gas phase therefore preferably has a melting point of 120 to 135° C., determined in accordance with DIN 51556:1963-07.

The pure isocyanate converted to the gas phase is preferably selected from aliphatic and/or aromatic diisocyanates, preferably from aromatic diisocyanates, in particular from naphthylene 1,5-diisocyanate.

In order to avoid excessively high temperatures and hence undesirable oligomerization reactions of the pure isocyanate, the process according to the invention is conducted under a negative pressure since this allows a reduction in the temperature required for evaporation of the pure isocyanate. The process is preferably conducted at a pressure of 0.1 to 5 mbar, preferably at 0.5 to 5 mbar, in particular at 1 to 5 mbar. This pressure is preferably already established in the working space prior to the supply of the isocyanate product stream in order to ensure that the pure isocyanate is substantially completely evaporated on the at least one heated roller 4 as soon as supply commences.

### Apparatus According to the Invention

A second subject of the present invention is an apparatus for the purification of a product stream from isocyanate synthesis.

This apparatus 1 comprises at least one working space 2, at least one product feed conduit 3 which is heatable and connected to the working space 2 for supplying the product stream into the working space 2, at least one heated roller 4 arranged in the working space 2, at least one isocyanate discharge conduit 6 connected to the working space 2 and optionally to the condenser 5 for discharging the isocyanate deposited on the condenser 5, at least one residue discharge conduit 7 connected to the working space 2 for discharging the residue collected on the at least one heated roller 4 and a negative pressure conduit 8 preferably connected to the working space 2 at the level of the condenser 5 for applying a negative pressure to the working space 2. The working space is preferably pear-shaped but can also have any other shape, for example a round shape.

Furthermore, the apparatus according to the invention comprises a condenser 5 which is arranged in the working space 2 and is a rotating cooled roller. At this roller is arranged at least one isocyanate removing device 9 for the continuous removal of the isocyanate deposited on the condenser 5.

The isocyanate product feed conduit 3 is preferably located above the at least one heated roller 4 and preferably ends immediately above said heated roller in order to ensure that the crude isocyanate supplied comes fully into contact with the at least one heated roller. In addition, the isocyanate product feed conduit 3 has an outer jacket for heating the isocyanate product feed conduit by means of a heating medium.

According to a first particularly preferred embodiment of the apparatus according to the invention, there is exactly one heated roller 4 in the working space 2. In this case, the isocyanate product feed conduit preferably ends—as viewed in the direction of rotation of the heated roller 4—immediately before the vertical axis of the heated roller 4.

According to an alternative particularly preferred embodiment of the apparatus according to the invention, there are exactly two heated rollers 4 and 4′ in the working space 2. These heated rollers are preferably arranged substantially parallel next to one another. The two heated rollers in this case are preferably not in contact with each other, i.e. there is an intermediate space between the two heated rollers. It is preferable here for the two heated rollers to be at the same height in the working space and to have opposite directions of rotation, i.e. the heated roller 4 rotates clockwise and the heated roller 4′ rotates counterclockwise or vice versa. In this embodiment, the isocyanate product feed conduit preferably ends immediately above the intermediate space formed by the two heated rollers.

At the at least one heated roller 4 there is preferably arranged a residue removing device 10, in particular a scraper, or at least one residue side discharge 10 for removing the residue collected on the at least one heated roller 4. The residue removing device 10 or the residue side discharge 10 is in this case preferably arranged before the point at which the isocyanate product feed conduit 3 ends. This ensures that the residue formed on the at least one heated roller 4 is substantially completely removed before this heated roller comes into contact with further crude isocyanate. If more than one heated roller 4 is arranged in the working space 2, each heated roller preferably has a residue removing device 10 or at least one residue side discharge—as described above.

The condenser 5 is preferably arranged above the at least one heated roller 4. This ensures that any residue falling off from the heated roller does not contaminate the pure isocyanate deposited on the condenser 5. In addition, it is preferable in accordance with the invention when the condenser 5 is located at the level of the negative pressure conduit 8 which is connected to the working space 2 and is for applying a negative pressure to the working space 2. This ensures that the pure isocyanate present in the gas phase first must pass the condenser 5 before it reaches the negative pressure conduit 8, as a result of which contamination of the negative pressure device with pure isocyanate as well as losses in yield are avoided.

According to a first preferred embodiment of the apparatus according to the invention, the working space 2 has no recesses. In this case it is preferable when the working space has a substantially pear-shaped form.

According to an alternative preferred embodiment of the apparatus according to the invention, the condenser 5 is arranged in a condensation chamber connected to the working space 2, wherein the connection between the working space 2 and the condensation chamber is preferably positioned laterally above the at least one heated roller 4 and opposite the product feed conduit 3. The working space 2 may in this case be of one-piece design. Alternatively, the condensation chamber may be attached to the working space 2 by means of a suitable fastening device 18, for example a clamp or the like.

The working space 2 preferably has an outer jacket 12 for heating the working space 2 by means of heat, in particular by means of steam and/or by means of oil. This ensures that the pure isocyanate present in the gas phase is not deposited on the walls of the working space 2 but instead substantially completely on the condenser 5.

### LIST OF REFERENCE SYMBOLS

1: apparatus for the purification of a product stream from isocyanate synthesis
2: working space
3: product feed conduit
4: heated roller 1
4′: heated roller 2
5: condenser
6: isocyanate discharge conduit
6′: drop shaft for isocyanate
7: residue discharge conduit
8: negative pressure conduit
9: isocyanate removing device 1
9′: isocyanate removing device 2
10: residue removing device 1
10′: residue removing device 2
12: working space outer jacket
13: product feed conduit outer jacket
13′: product feed conduit heating medium feed conduit
13″: product feed conduit used heating medium discharge conduit
14: residue collection device
15: isocyanate collection device
16: working space heating medium feed conduit
17: working space used heating medium discharge conduit
18: connection
V: vacuum

1. A process for the purification of a product stream from isocyanate synthesis, comprising the following process steps:
a) applying a negative pressure to a working space (2) by means of a negative pressure conduit (8) connected to the working space (2),
b) supplying the product stream to at least one heated roller (4) arranged in the working space (2) via a product feed conduit (3) which is heatable and connected to the working space (2),
c) converting the isocyanate present in the product stream to the gas phase on the at least one heated roller (4) and discharging the residue collected on the at least one heated roller (4) via at least one residue discharge conduit (7) connected to the working space (2) and
d) condensing the isocyanate converted to the gas phase at a condenser (5) arranged in the working space (2) and continuously discharging the isocyanate deposited on the condenser (5) via at least one isocyanate discharge conduit (6) connected to the working space (2) and optionally to the condenser (5),
characterized in that
the condenser (5) is a rotating cooled roller at which at least one isocyanate removing device (9) is arranged for the continuous removal of the isocyanate deposited from the gas phase on the condenser (5).

2. The process as claimed in claim 1, characterized in that the product stream has been obtained by one selected from the group consisting of phosgenation of an amine, phosgenation of an amine hydrochloride, phosgenation of a carbamate salt, the urea process, and reaction of an amine with dialkyl carbonates.

3. The process as claimed in claim 1, characterized in that the at least one heated roller (4) has a temperature which is 5 to 50 kelvin above the evaporation temperature of the isocyanate to be converted to the gas phase at the pressure prevailing in the working space (2).

4. The process as claimed in claim 1, characterized in that the residue formed on the at least one heated roller (4) is removed with the aid of a residue removing device (10).

5. The process as claimed in claim 1, characterized in that the isocyanate deposited continuously from the gas phase is removed from the at least one rotating cooled roller (5) with the aid of a scraper (9).

6. The process as claimed in claim 1, characterized in that the rotating cooled roller (5) has a temperature which is 5 to 30 kelvin below the melting point of the isocyanate converted to the gas phase at the pressure prevailing in the working space (2).

7. The process as claimed in claim 1, characterized in that the isocyanate converted to the gas phase has a melting point of 120 to 135° C., determined in accordance with DIN 51556:1963-07.

8. The process as claimed in claim 1, characterized in that the isocyanate converted to the gas phase is selected from aliphatic and/or aromatic diisocyanates, preferably from aromatic diisocyanates.

9. The process as claimed in claim 1, characterized in that the process is conducted at a pressure of 0.1 to 5 mbar.

10. An apparatus (1) for the purification of a product stream from isocyanate synthesis, comprising
at least one working space (2),
at least one product feed conduit (3) which is heatable and connected to the working space (2) for supplying the product stream into the working space (2),
at least one heated roller (4) arranged in the working space (2),
a condenser (5) arranged in the working space (2),
at least one isocyanate discharge conduit (6) connected to the working space (2) and optionally to the condenser (5) for discharging the isocyanate deposited on the condenser (5),
at least one residue discharge conduit (7) connected to the working space (2) for discharging the residue collected on the at least one heated roller (4) and
a negative pressure conduit (8) preferably connected to the working space (2) at the level of the condenser (5) for applying a negative pressure to the working space (2),
characterized in that
the condenser (5) is a rotating cooled roller at which at least one isocyanate removing device (9), is arranged for the continuous removal of the isocyanate deposited on the condenser (5).

11. The apparatus as claimed in claim 10, characterized in that in the working space at least two, heated rollers (4) are arranged substantially parallel next to one another.

12. The apparatus as claimed in claim 10, characterized in that at the at least one heated roller (4) is arranged at least one residue removing device (10), or at least one residue side discharge (10) for removing the residue collected on the at least one heated roller (4).

13. The apparatus as claimed in claim 10, characterized in that the condenser (5) is arranged above the at least one heated roller (4).

14. The apparatus as claimed in claim 10, characterized in that the condenser (5) is arranged in a condensation chamber connected to the working space (2), wherein the connection between the working space (2) and the condensation chamber is positioned laterally above the at least one heated roller (4) and opposite the product feed conduit (3).

15. The apparatus as claimed in claim 10, characterized in that the working space (2) has an outer jacket (12) for heating the working space (2), by means of steam and/or by means of oil.
